(19) United States
(12) Patent Application Publication
(10) Pub. No.: US 2013/0006289 A1
(43) Pub. Date: Jan. 3, 2013

(54) Ear Piercing Socket for Removable Starter Ear Rings

(76) Inventor: Sonya Harper, Broken Arrow, OK (US)

(21) Appl. No.: 13/171,719

(22) Filed: Jun. 29, 2011

(51) Int. Cl. A61B 17/34

(52) U.S. Cl. 606/188

(57) ABSTRACT

An ear piercing socket for use in piercing an ear includes a socket body having a generally tubular configuration sandwiched between an open body bottom and a closed body top. The socket body includes an interior surface defining an interior area, the open body bottom being in communication with said open body bottom. The interior area includes a configuration complementary to a configuration of an ear ring shaft so as to selectively and removably receive an ear ring shaft therein. The socket body top includes a pointed end extending away from the socket body, the socket body top being configured to puncture through a person's ear. The socket body top also includes a configuration to nest in the open end of an ear ring back. In some embodiments, the socket body may include an exterior surface having structures for releasing a medicament onto a person's ear.

### BACKGROUND OF THE INVENTION

This invention relates generally to ear piercing equipment and, more particularly, to an ear piercing socket having a hollow interior space for removably receiving a removable starter ear ring or other interchangeable ear rings.

The equipment and procedures for piercing ears so that a person may wear ear rings has been improving for hundreds or even thousands of years. Generally, an ear ring is quickly and forcefully inserted through a person's ear lobe and must remain there for a period of time, such as six to ten weeks, before it may be removed or replaced. It is specifically not recommended to remove or replace the ear ring for a period of time so that the hole does not reseal and to minimize the risk of infection.

The risk of infection may be reduced by applying anti-bacterial creams to each side of the ear and by not removing a starter ear ring for the recommended amount of time. Although assumably effective for their intended uses, the existing products and methods for piercing an ear and inserting start ear rings are inconvenient in that they require the ears to be treated with medicine and require that ear rings not be removed and replaced for several weeks.

Therefore, it would be desirable to have an ear piercing socket that may be inserted through a person's ear and into which a starter ear ring and, later, another ear ring may be selectively inserted and removed while the socket itself remains in the ear for the preferred number of weeks. It would also be desirable to have an ear piercing socket defining an open bottom into which an ear ring stud may be inserted and an interior nub for releasably securing the ear ring stud when inserted. In addition, it would be desirable to have an ear ring piercing socket having structures for discharging anti-infection medicine over an extended period of time.

### SUMMARY OF THE INVENTION

An ear piercing socket according to a preferred embodiment of the present invention includes a socket body having a generally tubular configuration sandwiched between an open body bottom and a closed body top. The socket body includes an interior surface defining an interior area, the open body bottom being in communication with said open body bottom. The interior area includes a configuration complementary to a configuration of an ear ring shaft so as to selectively and removably receive an ear ring shaft therein. The socket body top includes a pointed end extending away from the socket body, the socket body top being configured to puncture through a person's ear. The socket body top also includes a configuration to nest in the open end of an ear ring back. In some embodiments, the socket body may include an exterior surface having structures for releasing a medicament onto a person's ear.

Therefore, a general object of this invention is to provide an ear piercing socket for piercing a person's ear and into which an ear ring may be removably inserted and removed without removing the socket.

Another object of this invention is to provide an ear piercing socket, as aforesaid, that defines an open end and interior area configured to receive and releasably capture an ear ring shaft.

Still another object of this invention is to provide an ear piercing socket, as aforesaid, that includes a closed end having a pointed configuration suitable to pierce through a person's ear lobe.

Yet another object of this invention is to provide an ear piercing socket, as aforesaid, having a socket body having structures configured to slowly release a medicine applied thereto.

A further object of this invention is to provide an ear piercing socket, as aforesaid, that is easy to use and economical to manufacture.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is an exploded view of an ear piercing socket in use with an ear ring and ear ring back according to a preferred embodiment of the present invention;

FIG. 2a is a side view of the ear piercing socket as in FIG. 1;

FIG. 2b is a sectional view of the ear ring socket taken along line 2b-2b of FIG. 2a;

FIG. 3 is another side view of the ear ring socket as in FIG. 2a shown lodged in a person's ear;

FIG. 4a is a side view of an ear piercing socket according to another embodiment of the present invention;

FIG. 4b is a sectional view taken along line 4b-4b of FIG. 4a;

FIG. 4c is an isolated view on an enlarged scale taken from FIG. 4b;

FIG. 5a is a side view of an ear piercing socket according to another embodiment of the present invention;

FIG. 5b is a sectional view taken along line 5b-5b of FIG. 5a;

FIG. 5c is an isolated view on an enlarged scale taken from FIG. 5b;

FIG. 6a is an exploded view of the ear piercing socket as in FIG. 1; and

FIG. 6b is a sectional view taking along line 6b-6b of FIG. 6a.

### DESCRIPTION OF THE PREFERRED EMBODIMENT

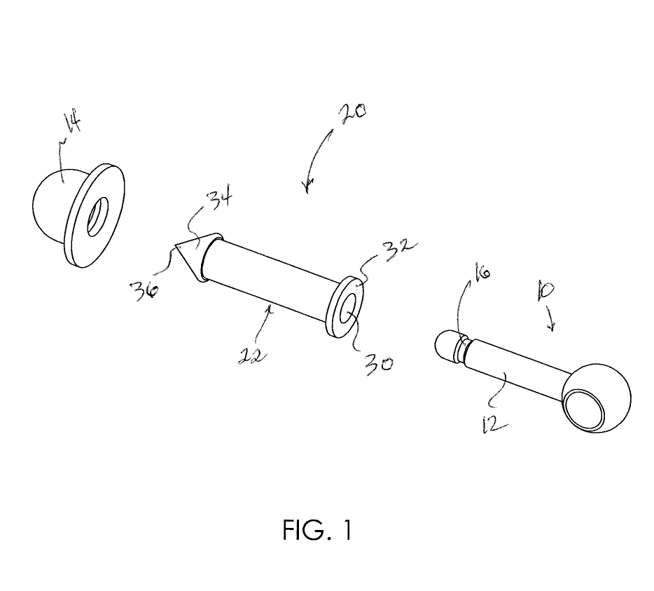

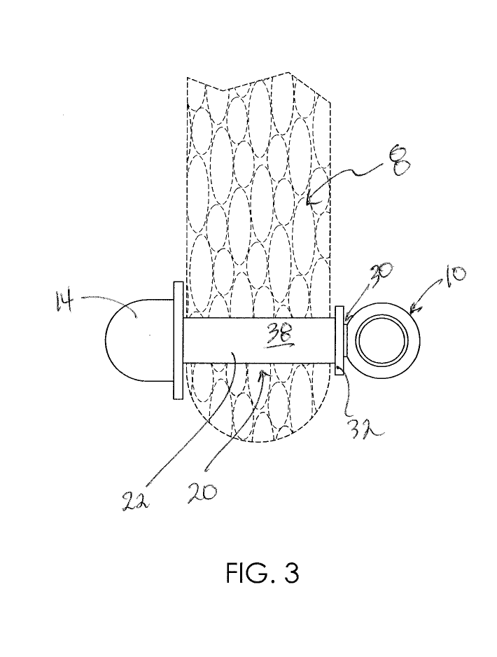

An ear piercing socket for use in piercing a person's ear according to a preferred embodiment of the present invention will now be described with reference to FIGS. 1 to 6b of the present invention. The ear piercing socket 20 is configured to be used with an ear ring 10 of a type having an ear ring shaft 12 with an ear ring back 14. In other words, the socket 20 is designed to work with an ear ring 10 that is inserted through one's ear and secured thereto with an ear ring back 14.

The ear piercing socket 20 includes a socket body 22 having a tubular configuration defining an open body bottom 30 and an opposed closed body top 34. The socket body 22 includes an inner surface 24 that defines an open interior area 26 in communication with the open body bottom 30 and includes a configuration so that an ear ring shaft 12 may be inserted through the body bottom 30 into the body interior as will be discussed more fully below. The interior area 26 presents a generally cylindrical configuration. A socket body rim 32 may surround the open body bottom 30 (FIG. 1). The closed body top 34 includes a pointed end 36 extending outwardly or away from the socket body 22.

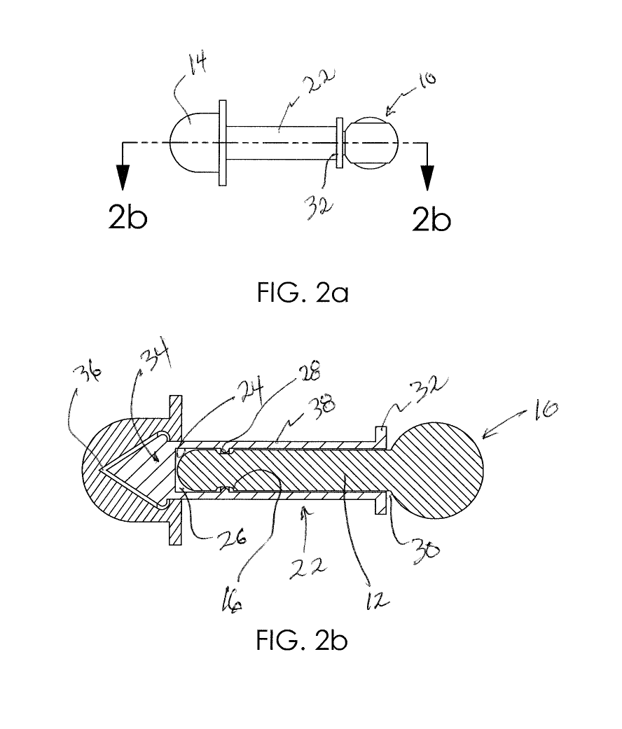

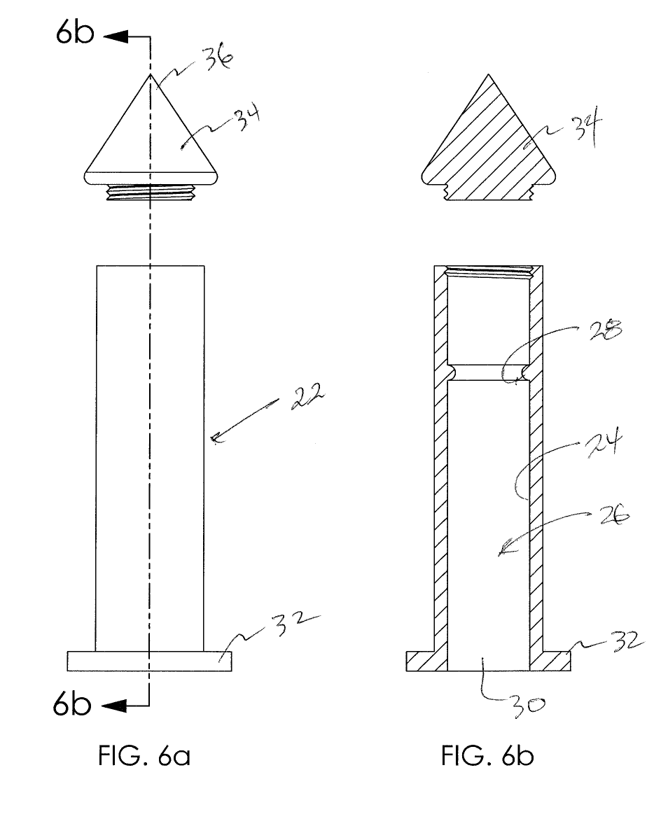

The closed body top 34 with pointed end 36 includes a configuration that is removably coupled to an ear ring back 14. Traditionally, an ear ring shaft 12 and ear ring back 14 are configured to fit together in a friction fit relationship—such as a snap fit—and that would be preferred in the case of the socket closed top 34 and ear ring back 14 (FIG. 2b). However, a threaded configuration may also be suitable. In some embodiments, the closed body top 34 may be removably coupled to the socket body 22, such as by a threaded attachment (FIGS. 6a and 6b). In this way, closed body top 34 members of different sizes and configurations may be interchangeably coupled to the socket body 22, perhaps for use with ear ring backs of different sizes or configurations.

The inner surface 24 of the socket body 22 may include a nub 28 substantially adjacent the closed top 34, the nub 28 being configured to removably capture the forward end of an ear ring shaft 12 when inserted into the interior area (FIG. 2b). Preferably, the ear ring shaft 12 defines at least one recess 16 positioned to mate with the nub 28 when the ear ring shaft 12 is inserted into the interior area 26. The nub 28 may be configured as a ring about the inner surface 24 of the interior area 26 of the socket body 22 and be situated essentially perpendicular to an imaginary longitudinal axis of the socket body 22.

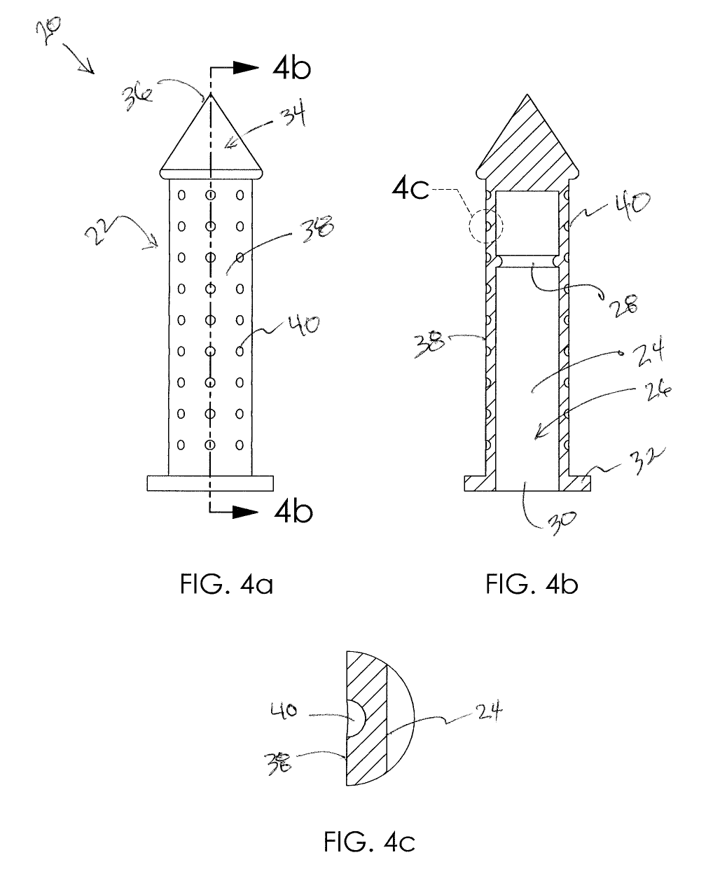

In some embodiments, the socket body 22 includes a configuration specifically configured to dispense a medicament over an extended period of time, such as anti-bacterial or anti-infection cream that would inhibit infection at the site of the newly punctured ear lobe 8. With specific reference to FIGS. 4a to 4c, the exterior surface 38 of the socket body 22 may define a plurality of recesses 40 or pits configured to retain a medicament when the socket body 22 is inserted through an ear lobe. With the plurality of recesses 40, some of the medicament may be immediately smeared over the punctured ear surface while additional medicament may remain in some recesses 40 and be absorbed by the punctured ear tissue over a period of days or weeks.

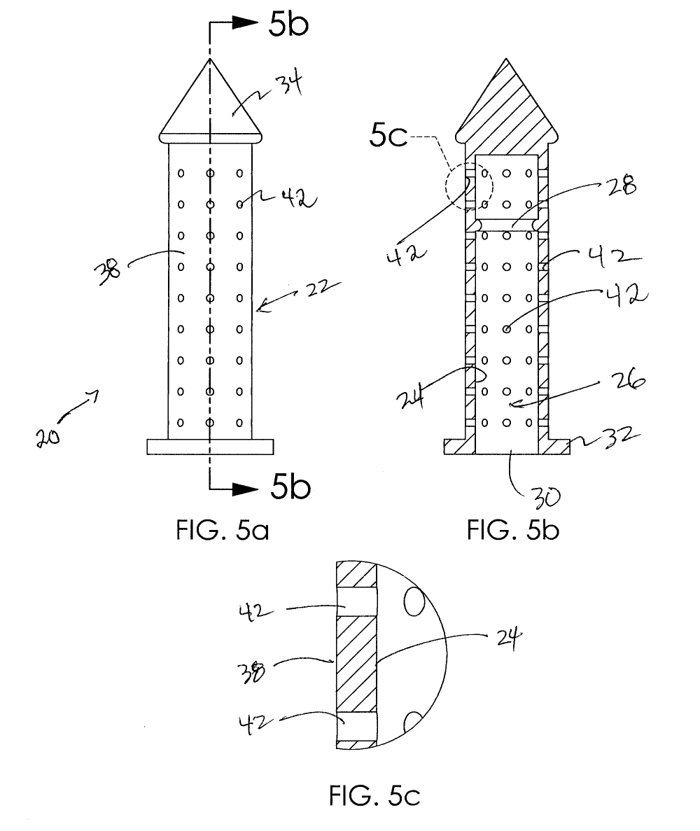

With specific reference to FIGS. 5a to 5c, the socket body 22 may define a plurality of spaced apart apertures 42 that communicate the interior area 26 with an area outside the socket body 22. In this embodiment, a medicament, such as an antiseptic cream may be initially inserted into the interior area 26 prior to insertion of the socket body 22 through an ear lobe 8. Then over a period of days or weeks, the antiseptic cream is expected to ooze through the plurality of apertures onto the ear lobe, especially as ear ring shafts 12 are inserted and removed from the interior area.

In use, an ear piercing socket 20 may be inserted through the lower ear lobe 8 of a person's ear, such as by using a traditional ear piercing gun or a specialty ear piercing apparatus as may be appropriate for use with the present invention. More particularly, the closed top 34 of the piercing socket 20 may be positioned against the ear lobe 8 at the desired position, the pointed end being configured to pierce all the way through the ear lobe 8 when the ear piercing gun is actuated. The act of vigorously inserting the ear piercing socket 20 results in the socket body 22 being left extending through the ear lobe 8. Preferably, an ear ring shaft 12 is positioned inside the socket body interior area 26 prior to inserting the socket body 22 although it may be inserted later. The ear ring back 14 may be coupled to the socket body closed top 34 following insertion so as to prevent the socket body 22 from inadvertently slipping out of the ear lobe. The ear ring shaft 12 is securely held inside the interior area 26 by the nub 28 nesting with a recess 16 defined by the ear ring shaft 12. As desired by a user, the ear ring shaft 12 may be removed from the interior area 26 and replaced with a then preferred ear ring shaft 12 so that the starter ear ring need not be worn for the 4-6 weeks following piercing of the ear lobe.

In the case of the embodiments capable of carrying an antiseptic medicament, a cream may be spread upon the socket body exterior surface 38 or within the interior area 26 such that a pierced ear may receive the medicine repeatedly throughout the typical 4-6 weeks in which a starter rear ring 10 must normally not be removed. However, the product design of the present invention enables starter ear rings to be interchanged throughout the normal starter period without risking a buildup of infection or other undesirable maladies.

It is understood that while certain forms of this invention have been illustrated and described, it is not limited thereto except insofar as such limitations are included in the following claims and allowable functional equivalents thereof.

1. An ear piercing socket for use with a removable starter ear ring having a shaft and a back, said ear piercing socket comprising:
a socket body having a tubular configuration sandwiched between an open body bottom and a closed body top, said socket body having an interior surface defining an interior area in communication with open body bottom;
wherein said interior area is configured to selectively receive the shaft of the starter ear ring; and
wherein said closed body top includes a pointed end extending away from said socket body.

2. The ear piercing socket as in claim 1, wherein said closed socket top includes a configuration that is removably coupled to the ear ring back.

3. The ear piercing socket as in claim 1, wherein said interior surface of said socket body includes a nub having a configuration to frictionally capture a forward end of the ear ring shaft.

4. The ear piercing socket as in claim 3, wherein said nub is substantially adjacent said socket closed end.

5. The ear piercing socket as in claim 4, wherein:
said interior surface of said socket body has a cylindrical configuration; and
said nub is a ring extending about said interior surface perpendicular to an imaginary longitudinal axis defined by said socket body.

6. The ear piercing socket as in claim 1, wherein said closed body top is selectively removable from said socket body.

7. The ear piercing socket as in claim 6, wherein said closed body top is threadably coupled to said socket body.

8. The ear piercing socket as in claim 1, wherein said socket body includes an exterior surface defining a plurality of recesses configured to retain a medicament.

9. The ear piercing socket as in claim 1, wherein said socket body includes an exterior surface defining a plurality of apertures spaced apart from another, each aperture being in communication with said socket body interior area.
